(19) United States
(12) Patent Application Publication
(10) Pub. No.: US 2007/0232923 A1
(43) Pub. Date: Oct. 4, 2007

(54) Active thermal management for ultrasound catheter probe

(76) Inventor: Bhushan Shanti Asuri, San Diego, CA (US)

Correspondence Address: Law Offices of Terry McHugh, 101 First Street - PMB 560, Los Altos, CA, 94022, US

(21) Appl. No.: 11/715,768

(22) Filed: Mar. 8, 2007

Related U.S. Application Data
(60) Provisional application No. 60/780,188, filed on Mar. 8, 2006.

(51) Int. Cl. A61B 8/14

(52) U.S. Cl. 600/459; 600/463

(57) ABSTRACT

In accordance with the invention, an ultrasound imaging device includes a fluid flow path between a distal end having ultrasound transducers and a proximal end that is electrically coupled to the distal end to enable the exchange of electrical signals. Cooling fluid within the flow path transfers heat generated by the ultrasound transducers and other heat-generating components located at the distal end. The heat flow path is one element of an active thermal management sub-system, which may also include a Thermo Electric Cooler (TEC). The active thermal management sub-system may alternatively or additionally include a heat sink to transfer thermal energy. A temperature monitor may be used for the embodiment in which the sub-system dynamically provides thermal regulation.

### CROSS REFERENCE TO RELATED APPLICATION

This application claims priority from co-pending provisional application Ser. No. 60/780,188, filed Mar. 8, 2006.

### TECHNICAL FIELD

This invention relates to thermal management of dense ultrasonic probes, in particular, for probes used for diagnostic imaging.

### BACKGROUND ART

Ultrasound is based on the transmission of sound waves through the human body and recording the pattern of received echoes. The timing of the echo determines the depth of the object producing the echo, and its strength determines the contrast that the echo-producing object has with respect to its environment.

Typical ultrasound systems use a variety of means for scanning the transmitted energy through a volume or area of interest. This extended ultrasound map of the body is designated by various letters to signify the type of scanning: B-mode refers to the method of scanning a slice of the human body, and volume scanning refers to the scanning of a whole volume of the body.

The transmit and receive beams can be scanned by the use of ultrasound phased arrays that contain many piezoelectric elements. During transmit, the relative phase and amplitude of the signal emitted from each element is chosen to form and focus a transmit beam toward the point of interest. A receive beam is formed and focused similarly by delaying the received signal from different elements, scaling them appropriately and summing the received signals. Conventional B-mode scanning employs one-dimensional (1D) transducer arrays to capture tomographic image slices of areas of the human body. The phased array in this method scans the beam only in the azimuth direction. A slightly advanced form of B-mode scanning is realized with 1.5D arrays, where multiple adjacent rows of 1D array are combined to control the beam width and steering in the elevation direction.

Volume scanning is desirable because, by forming and collecting volumes rather than slices, it presents more complete structural information, and image slices that are derived from those volumes are viewed in a more complete anatomic context than slices derived from B-mode. Image volumes may be acquired by mechanically scanning 1D arrays to acquire multiple adjacent B-mode image slices and assembling them for display. However, mechanical scanning is cumbersome and slow, however, and electronic scanning is preferred. Electronic volumetric scanning requires two-dimensional (2D) arrays to steer the transmit beam in two dimensions, whereas B-mode scanning requires the transmit beam to sweep with 1D arrays in only one dimension.

The spatial resolution of 2D arrays is determined by the aperture size. Element pitch is determined by the acoustic wavelength of the ultrasound signal. These constraints often lead to upwards of 2000 elements being required for fully populated 2D arrays with resolution in the elevation direction comparable to the azimuth resolution of traditional B-mode scanners.

The ultrasound transducer is typically composed of a piezoelectric material appropriately machined into individual elements that are connected to a specially constructed cable. This cable, which might contain hundreds of micro-coax wires or ribbon wires, carries signals from the transducer and delivers drive signals from the drive electronics back to the transducer.

The cables for ultrasound systems demand a flexible mechanical design to prevent ergonomic injuries to ultrasound technicians. For example, the cable that connects the transducer to the system console must be flexible and lightweight so as not to impede the scanning that is performed by hand and not to fatigue or unduly stress or, in the long term, cause injury to the sonographer during the time of the scanning procedure. These demands generally trade-off with the number of micro-coax wires that a cable can support, since adding more wires to a cable bundle makes the cable stiffer and more difficult to maneuver. Furthermore, tighter packing of multiple cables greatly impacts the electrical performance of the wire bundle. These factors make the design and manufacture of 1000-wire cables extremely costly and cumbersome. Finally, a large cable bundle makes it difficult to shrink the size of the probe for interventional applications. These applications would use catheter size probes.

Another trend is that the electrical impedance of an individual element rises as its size is reduced. The size reduction is particularly severe for true 2D arrays, where the piezoelectric is diced in both azimuthal and lateral directions to form rows and columns of elements. The higher impedance decreases the strength of the signal transferred from transducer to cable, unless special measures are taken. These measures could include using wire bundles with lower capacitance per unit length and/or terminating the cable at the transducer end with an impedance-matching circuit. These choices place additional demands on the conventional system design, but various conventional ultrasound systems employ one or both of them. Being passive, they are relatively straightforward to implement and have limited effect on overall system performance.

Other measures include the incorporation of active electronics in the transducer scan head, so as to enable driver circuits to actively induce currents into the transducer cables to reduce or eliminate losses in those cables.

Another set of solutions to accommodating the larger numbers of elements in 2D arrays centers around the use of active multiplexing of signals from various transducer elements into a single wire. These methods include the use of time-domain multiplexing, frequency-domain multiplexing, and a method of additively combining the signals prior to transmitting them on the micro coax wire. In practice, each of these methods used independently is unlikely to be able to support very large arrays. Using a combination of methods to increase the multiplexing capacity of these systems requires a large amount of space (area) and/or power (heat).

Accordingly, the use of 2D arrays for volume-scanned ultrasound phased array systems faces various challenges, the most significant being the need to support a large number of array elements and, in turn, a very large cable bundle. While several individual means of multiplexing have been proposed, each of these is severely limited in the number of elements it can support while maintaining reasonable power dissipation and area consumption.

A portion of the electrical power delivered to the transducer is converted to heat because of the limited efficiency of the electro-acoustic conversion. The use of integrated circuits in the proximity of the transducer elements creates another source of heat that can raise the temperature of the catheter assembly in which the probe is located. The thermal dissipation from various sources limits the transmit power of the probe and the ability to integrate electronics in proximity to the probe elements.

Thermal management is a key challenge in the design of dense ultrasound probes. Thermal dissipation in the probe and/or associated electronics can cause undesirable temperature rise. Moreover, regulations restrict the in-vivo maximum temperature of the probe tip to 40° C. Most of the epoxies commonly used in ultrasound arrays have Curie temperatures of 65° C., which thus establishes the maximum temperature at any point within the probe.

### SUMMARY OF THE INVENTION

The invention is related to active thermal management of ultrasound probes. The invention allows higher transmit powers and more complex integrated circuits, potentially improving signal quality while maintaining the temperature of the transducer and catheter body within operational limits.

The ultrasound imaging device includes a distal end having ultrasound transducers, a proximal end electrically coupled to the distal end to enable the exchange of electrical signals, and a fluid flow path between the proximal end and the distal end. Cooling fluid within the flow path transfers heat generated by the ultrasound transducers and other heat-generating components (e.g., integrated circuitry) of the distal end.

The fluid flow path forms one element of an active thermal management sub-system. In one embodiment, the sub-system also includes at least one Thermo Electric Cooler (TEC) positioned within the distal end to transfer heat. A TEC is a heat pump that transfers heat when a voltage is applied to dissimilar materials. A typical TEC includes an array of p-type and n-type semiconductor elements that provide a temperature difference when a voltage is applied. There are a number of possibilities for the use of one or more TECs. As one possibility, the ultrasound transducers of the distal end may be in contact with a fluid bath that is separated from the cooling fluid of the flow path by a TEC. Thus, the fluid bath is in thermal transfer engagement with the fluid flow path via the TEC. Alternatively or additionally, heat-generating components at the distal end may be in contact with a TEC that transfers the heat directly to the fluid within the flow path.

The active thermal management sub-system may also include a heat sink in contact with the cooling fluid. The heat sink may be a passive component of the sub-system, but is used to define a thermal flow path in addition to the fluid flow path. The heat sink may initiate the thermal flow path, such as when the heat sink is in direct contact with heat-generating components at the distal end. Alternatively, when the cooling fluid returning from the catheter tip is at an elevated temperature, the heat sink may be downstream and may be used to provide cooling. The heated fluid may be directed through the heat sink, such as in the embodiment in which the heat sink is a metal plate having copper piping. Fins on the metal plate may be included to allow convective cooling of the plate. The copper piping embedded in one face of the metal plate transfers heat to the plate, with the heat then being transferred to the fins. It is also possible to provide forced air cooling of the heat sink, such as the use of a fan directed along the fins of the heat sink. In some applications, it is possible for the heat sink to carry the heat to the bloodstream of the body into which an ultrasound image is to be formed. Then, the blood functions as a natural coolant. This embodiment may be useful for devices that are placed in anatomical regions or body cavities with robust blood flow.

The proximal end of an ultrasound system that includes the active thermal management may be designed to allow fluid inlet and outlet, while supporting both electrical cables that connect to the probe and the means for mechanically manipulating the distal end. The mechanical means may be one or both of steering wires and rotational shafts. One or more pumps and valves may be used to regulate the flow rate and the flow direction of the cooling fluid, which may be castor oil. The rotational shaft may contain wires that are insulated and electrically isolated from the cooling fluid, so as to safely carry signals to and from the probe. In another embodiment, a single lumen extends between the proximal end and the distal end, with the flow of direction being regulated.

The active thermal management sub-system may be aided by the presence of a thermally conductive, but low electrically conductive, epoxy barrier around the transducer assembly. This epoxy barrier may prevent the cooling fluid from contacting the electronics and/or the transducer elements. The barrier may need to have a sufficiently high Curie temperature to withstand the maximum temperature within the particular environment.

An alternative embodiment of the invention is one in which constant fluid circulation is avoided. Instead, small volumes of fluid are dispensed in batches. Effective cooling is possible if the temperature of the fluid is sufficiently low. In this case, the lack of heat capacity of discontinuously dispensed fluid may be overcome by the low temperature of the dispensed volume. As one possibility, an electrical cooling system may be employed, such as a refrigeration device for the dispensed cooling fluid.

Yet another possible component of the active thermal management sub-system is a temperature monitor. As one possibility, the temperature monitor may be a thermistor. Particularly if the electronic cooling device is employed, the temperature at the catheter tip may be maintained within a target range. This may be accomplished by varying the rate at which fluid is pumped through the fluid flow path. Additionally or alternatively, a TEC may be controlled to provide temperature regulation within a particular range.

### BRIEF DESCRIPTION OF THE DRAWINGS

FIG. 1 illustrates one embodiment of a thermal management sub-system for use in ultrasound probes.

FIG. 2 illustrates an embodiment of the proximal tip that supports an ultrasound probe with active thermal management.

FIG. 3 illustrates an alternative embodiment of the active thermal management sub-system.

FIG. 4 illustrates an embodiment of the invention where the fluid used for active cooling does not flow around the ultrasound transducer.

FIG. 5 illustrates an embodiment of the invention where a heat sink in contact with blood carries the heat away from the transducer.

FIG. 6 illustrates the method of using active thermal management in conjunction with an ultrasound system.

FIG. 7 illustrates an embodiment of the invention where the fluid does not need to circulate in a closed path.

FIG. 8 illustrates an embodiment of the active thermal management sub-system used in conjunction with the a rotating one-dimensional array for creating 3-D ultrasound images.

FIG. 9 illustrates a time gated imaging system used in conjunction with the active thermal management to reduce the power dissipated in the ultrasound probe.

### DETAILED DESCRIPTION

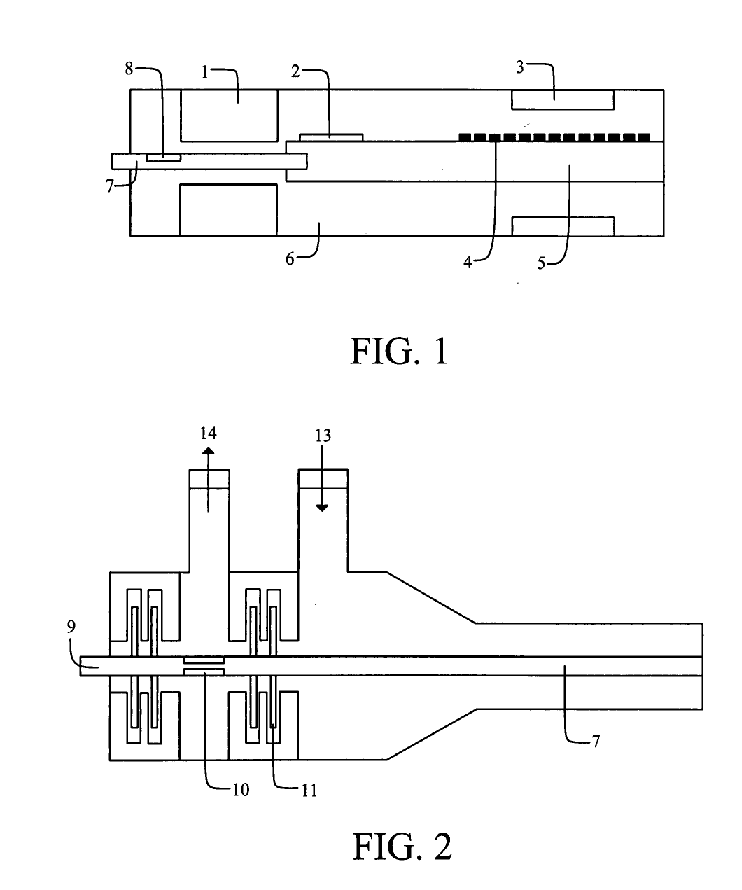

FIG. 1 illustrates a particular embodiment of the invention. A Thermo Electric Cooler (TEC) 1 transfers heat from an integrated circuit 2 and transducer elements 4 to the circulating fluid. A hollow mechanical shaft 7 contains an opening 8 to carry the fluid to the proximal end (not shown). The transducer and integrated circuit are placed in a fluid bath 6. The fluid in the bath enables acoustic coupling of the transducer elements to an acoustic lens 3. The mechanical shaft connects to a flex Printed Circuit Board (PCB) 5 that may be stiffened to provide mechanical rigidity. The fluid within the bath 6 resides both above and below the PCB, as viewed in FIG. 1.

The TEC 1 is used as a heat pump. A conventional TEC is a solid state heat pump that operates on the Peltier effect. That is, heating or cooling occur when electric current passes through two dissimilar conductors, such as by the application of a voltage to ends of a material stack formed of alternating p-type and n-type semiconductors. In FIG. 1, the TEC is located to remove heat from the fluid within the bath 6. The transfer of heat may be to a heat sink, for example.

FIG. 2 illustrates the proximal end of an ultrasound probe with active thermal management that supports a rotating array at its tip. A rotational shaft 7 carries fluid from the distal tip which is expelled through openings 10 to an outlet valve 14. An inlet valve 13 directs the coolant towards the distal tip. Rotational fluid seal 11 provides hydraulic isolation between the inlet and outlet chambers. The rotational shaft is further coupled to a mechanical rotational system.

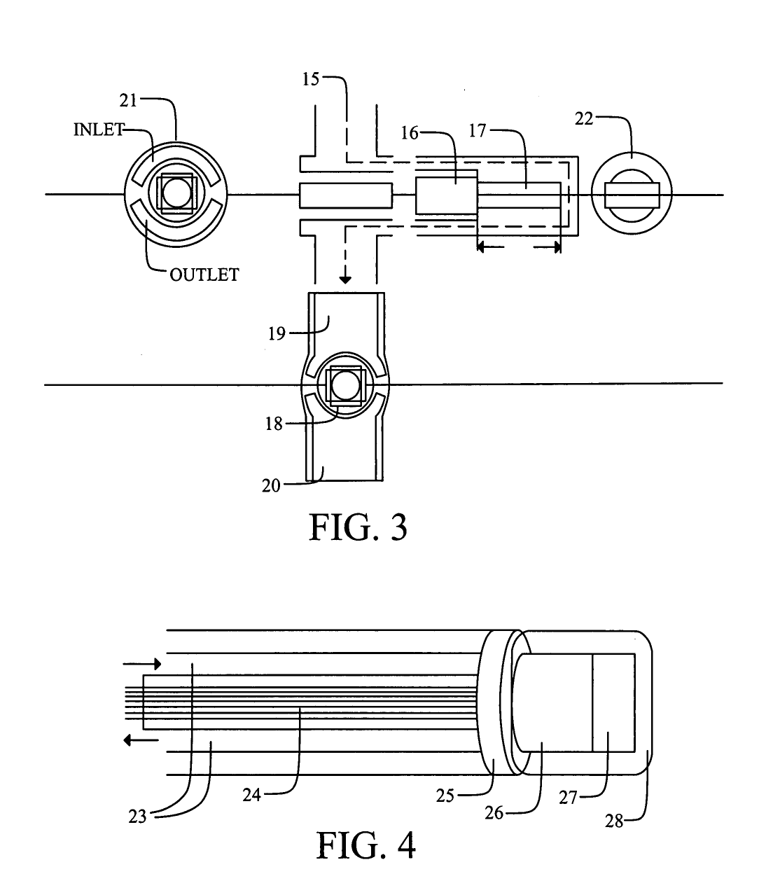

FIG. 3 illustrates the various views of an alternate embodiment of the active thermal management system. For explanation, the views include each end view, a side view, and a bottom view. Here, the fluid path is indicated by dashed line 15, which flows through an inlet valve 19, through a catheter lumen, over the distal tip transducer and integrated circuit assembly 17 to the outlet 20. The internal lumen contains a mechanical shaft connected to the distal tip assembly and electrical conductors 18 from the ultrasound probe. Short axis cross sectional views present illustrations of the lumen at the proximal end 21 and distal end 22.

FIG. 4 illustrates an embodiment of the invention in which an annular heat sink 25 carries heat away from an integrated circuit 26 and an acoustic stack 27 and delivers the heat to a circulating coolant 23. A lumen in the catheter 24 carries the mechanical shaft connected to the distal tip and the electrical conductors connected to the distal probe. An acoustic window 28 couples ultrasound energy from the transducer. In FIG. 4, the annular heat sink may be in direct contact with blood to dump a portion of the heat directly into the blood stream.

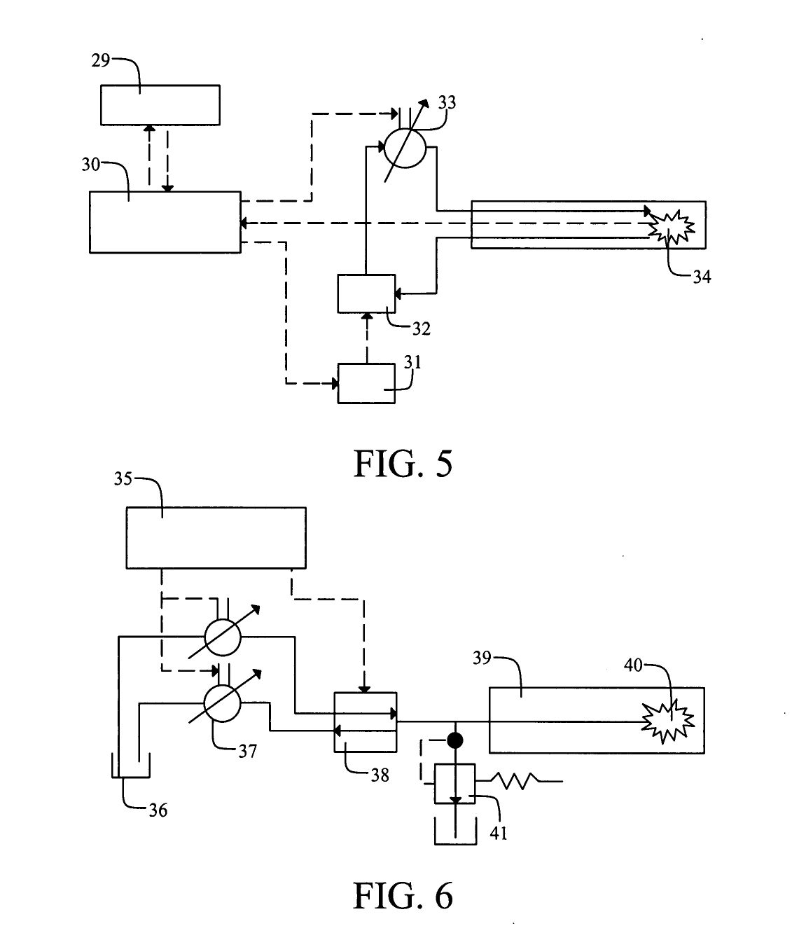

FIG. 5 illustrates a method of controlling the fluid flow using a variable capacity pump 33. Temperature information from the distal tip 34 is sent to a thermal control unit 30 which in conjunction with a main ultrasound system 29 determines the pumping rate and the drives the Thermo Electric Cooler 31 that is in contact with a heat sink 32. The thermal control may either increase fluid pumping rate, or reduce the inlet fluid temperature, or both, if the temperature of the tip starts to rise.

FIG. 6 illustrates an embodiment of the invention in which a bidirectional pump 37 is utilized instead of circulating a coolant in the lumen 39. This embodiment enables the use of one lumen instead of two, thereby allowing reduction in the size of the catheter. Coolant from a reservoir 36 is directed into or out of the lumen by an electro-mechanical control unit 35 which controls the bidirectional pump 37 and valve 38. The coolant flows through the lumen to the active distal tip 40. A bleeder valve 41 is attached to the line to bleed any excess fluid.

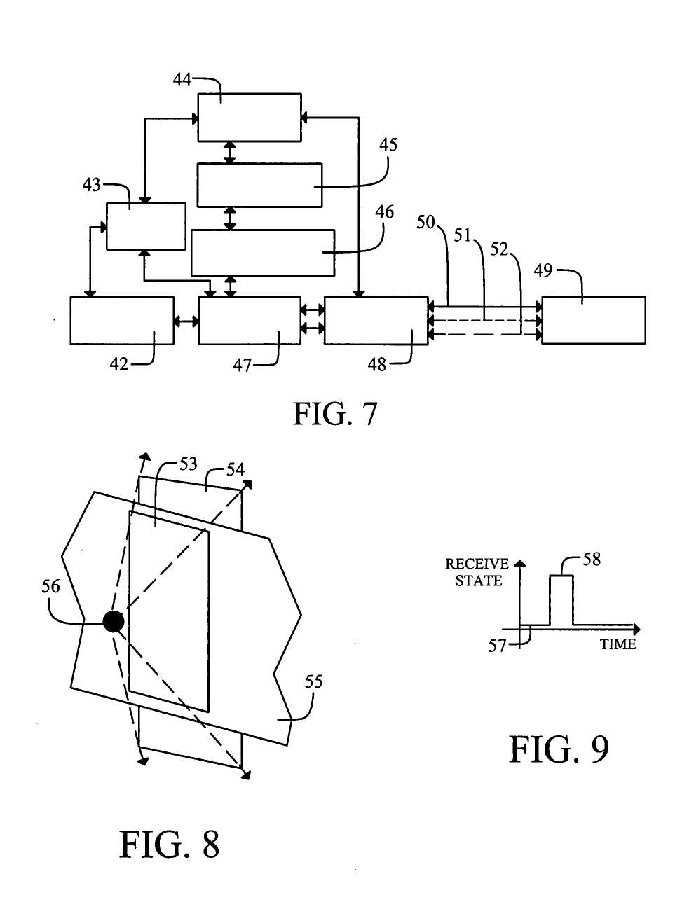

FIG. 7 illustrates the method of interfacing the active thermal management system with the base unit. Here, the distal tip 49 has a fluid coupling 50, mechanical coupling 52 and electrical coupling 51 to the base unit. The base unit software 44 controls the mechanical sub unit 42. The base unit software receives distal tip temperature information from the electrical sub unit 47 through the thermal control sub unit 43. The electrical sub unit provides access to the probe electrical signals to an analog signal acquisition 46. The distal tip temperature information is used to drive a fluid sub unit 48 which controls the fluid flow rate and inlet fluid temperature. Image processing sub unit 45 delivers processed information for display.

FIG. 8 illustrates a method of axial ranging to limit the power consumption of the ultrasound probe. Here, known methods of image segmentation are used to determine planes 53 and 54 such that the blood tissue interface or any other anatomical interface of interest 55 lies between said planes. The transducer location 56 determines the time interval over which the imaging system needs to remain “on” for each image line. One example of such time gating is shown in the insert, where for one of the image lines the receive stays in its “off” state 57 and is turned “on” 58 for a brief interval corresponding to the time of reception of signals from anatomical points that lie between the planes of interest. By turning the receive to the “on” state during a small period, this method reduces the power consumption of the ultrasound probe.

As described in the various embodiments above, the proximal end of an ultrasound probe with active thermal management may be designed to allow fluid inlet and outlet, while supporting electrical cables that connect to the probe and while supporting means for mechanically manipulating the distal end. The mechanical means may include one or both of steering wires and rotational shafts. The inlet valve, such as the valve 19 in FIG. 3, may be driven by a suitable pump. The fluid injected into the inlet valve flows to the distal tip 22 through a channel or channels in the catheter body. The fluid then returns from the distal tip through the hollow shaft that extends along the body of the catheter. The shaft may contain wires that are insulated and electrically isolated from the cooling fluid, such as water or castor oil. FIG. 4 shows an array of such wires extending from the transducer to carry signals to and from the probe.

The shaft may be mechanically coupled to the outlet chamber which is isolated from the inlet chamber with a high flow resistance coupling joint. The fluid may then be flushed from the outlet valve through an opening in the fluid-resistant wall of the shaft. Alternatively, the fluid flow may be in the opposite direction, such that the input is through the hollow shaft and the output is through the extruded channel or channels. In some embodiments, the shaft may be used to rotate the probe mounted at the distal end. The rotational shaft is coupled through another fluid-resistant coupling joint to electrical connectors that are situated outside the fluid chambers. It should be noted that the shaft, depending on the desired implementation of the invention, may not accommodate rotational coupling to the distal tip. In this case, the input and output chambers may be separated by a simple water-tight valve.

Another method for active thermal management involves circulating fluid coolant around the active distal tip. In this case, the flowing coolant directly contacts the active elements in the ultrasound probe, such as the transducers and any integrated circuitry. The extrusion is connected to outlet and inlet valves at the proximal end. The cooling fluid circulates in a loop in the extrusion. The fluid path and the extrusion comprises an annular opening that is separated into an inlet and outlet section with a barrier that runs along the catheter axis. The barrier stretches to a point proximate to the distal tip of the catheter. The distal tip contains the transducer assembly. Fluid from the inlet section of the annular opening flows around the transducer assembly to the outlet section of the annular opening.

The active thermal management sub-system may be aided by the presence of a thermally conductive epoxy barrier around the transducer assembly. In this embodiment, the epoxy should have a low electrical conductivity. The epoxy barrier may be used to prevent the cooling fluid from coming in direct contact with the electronics and/or the transducer elements. The epoxy barrier may need to have a sufficiently high Curie temperature to withstand the maximum temperatures encountered in the particular application of the ultrasound probe.

It should be noted that the active thermal management sub-system can be applied to ultrasound probes irrespective of whether the probes contain an integrated circuit at the distal tip. If no integrated circuit is included, the active thermal management controls temperature rise due to loss of acoustic energy in the backing material and may reduce heating on the skin line that is a result of high frequency content in the ultrasound transducer output.

In the embodiment of FIG. 5, the sub-system includes a heat sink 32. The heat sink may be a passive component that is in physical contact with active elements in the probe. The heat sink carries the heat to a cooling fluid that is continuously circulated. This embodiment may be useful in preventing the accidental introduction of air bubbles in the distal tip. In this embodiment, the extrusion may be divided into inlet and outlet channels. The fluid carries heat away from the annular heat sink. The heat sink isolates the distal tip from the cooling fluid path while at the same time providing a thermal path for heat to flow from the distal tip. A central lumen in the extrusion contains the conductors that connect to the distal tip.

The heat sink can also dump the heat in the bloodstream of the body being imaged. In such a situation, the blood is used as a natural coolant. This embodiment may be useful for devices that are placed in anatomical regions or body cavities with robust blood flow.

FIG. 5 and other embodiments also show the use of a TEC. Whether a passive heat sink or a TEC is used, the heat from the cooling fluid may be transferred to the surrounding medium, such as air. A pump constantly circulates cooling fluid around the catheter lumen. The fluid returning from the catheter tip may be at an elevated temperature, but the heat sink or a TEC may be used to transfer the heat from the fluid. A possible example is a passive heat sink which is implemented as a metal plate with copper piping. The metal plate may include fins which allow conductive cooling of the plate. The copper piping embedded on one face of the metal plate transfers heat to the metal plate and subsequently to the fins. Alternatively or additionally, the heat sink arrangement may consist of a fan-cooled fin structure. The fan cooling would allow forced convection cooling of the metal plate and therefore the fluid. In an alternative embodiment, a TEC is used to cool the heat sink or pipes carrying the cooling fluid.

Again referring to FIG. 5, the pump 33 may be operated by the thermal control unit 30 in a non-continuous manner. That is, rather than a constant fluid circulation, small volumes of fluid may be dispensed in batches. Effective cooling may still be possible, if the temperature of the fluid is sufficiently low. In this case, the lack of heat capacity of discontinuously dispensed fluid may be overcome by the low temperature of the dispensed volume. Thus, the thermal control unit 30 may include a refrigeration system. In a more complete application, the thermal control unit also includes a temperature sensor, such as a thermistor. Temperature sensing is also significant for applications in which the pump and/or any valves are controlled on the basis of maintaining the catheter tip within a target range of temperatures. Thus, the pump 33 of FIG. 5 may be controlled to vary flow rate on the basis of temperature. As another possibility, the TEC 31 may be controlled on the basis of current temperature.

The cooling fluid need not be circulated around the catheter body. Alternatively, the fluid may be expelled at any point in the path after the fluid has come into contact with the active elements. When expelled, the fluid may be directed outside the catheter either in the body or outside the body. As another alternative, forced fluid cooling can pump in and pump out fluid through the same fluid path. A bidirectional pump 37 is shown in FIG. 6. In this case, the fluid does not need a closed path for flow. A reservoir 36 stores the cooling fluid. A variable capacity pump drives fluid into one lumen 39 of the device. The fluid flow direction is controlled by the electrically driven fluid valve 38. In the next phase of its action, the electrical control opens the valve to the outbound direction. The fluid is removed by the pump. The fluid may then be cooled before return to the reservoir. The control unit may monitor the amount of fluid in the reservoir to ensure that there is no fluid accumulation within the lumen of the device. A bleeder valve 41 is shown in FIG. 6 for use to remove any excess fluid from the lumen, if the pressure in the lumen exceeds a safe value.

The ultrasound system uses electrical signals to form images, as is well known in the art. Electrical signals from the distal tip carry radio frequency (RF) information from the ultrasound probe. This RF information may be processed with an analog front end by an image processing end rendering unit. The processed image is routed through the system software to be displayed on a user console. In the present invention, the system software may also directly monitor the temperature of the distal tip through electrical signals that carry temperature-related information.

Such information can be generated by various known means, including a thermistor placed at the distal tip. Other means can include an integrated circuit temperature sensing means like a PTAT (proportional to absolute temperature) voltage source. Alternate means could include indirect monitoring of the distal tip temperature by monitoring the temperature of the outbound fluid. Based on the temperature at the distal tip, the system software may control the electrical, mechanical and fluid coupling sub-units. In one embodiment, the system software may temporarily power down the transducer if the temperature rises to unacceptable levels.

Another embodiment of the invention concerns the powering down of receive circuits at the distal tip to monitor signals over a fixed axial range. This feature may be used in conjunction with edge detection software. One example of the use of this method is in the imaging of heat chambers. Often, medical practitioners are interested in observing the motion of the heart wall. This means that it is sufficient to image the axial range corresponding to the location of the heart muscle-blood boundary over a complete cardiac cycle. This axial range can be determined from full volumetric images using known image edge detection techniques. Once the axial range is known, it can be mapped to a time window by using information about the speed of sound. The receive circuits may be activated only during the time window. The time-gated operation of the receive circuits reduces the average power dissipation. The reduction in power dissipation lessens the burden on any thermal management system. Conversely, the same thermal management system can now be used with more complex or better performing receive circuits.

What is claim is:

1. An ultrasound imaging device comprising:
a probe that includes a distal end having ultrasound transducers and a proximal end electrically coupled to said distal end for conducting electrical signals; and
a fluid flow path between said proximal end and said distal end, wherein a coolant within said fluid flow path is in thermal transfer engagement with said ultrasound transducers, said fluid flow path being configured to transfer heat from said ultrasound transducers.

2. The ultrasound imaging device of claim 1 wherein said distal end includes at least one integrated circuit that is in thermal transfer engagement with said coolant within said fluid flow path.

3. The ultrasound imaging device of claim 1 further comprising a Thermo Electric Cooler (TEC) positioned within said distal end to transfer heat to said coolant within said fluid flow path.

4. The ultrasound imaging device of claim 1 wherein said distal end includes a fluid bath in which said ultrasound transducers are in contact.

5. The ultrasound imaging device of claim 4 wherein said fluid bath is separated from said fluid flow path by a partition that includes at least one TEC.

6. The ultrasound imaging device of claim 1 wherein said ultrasound transducers define an ultrasound phased array and wherein said distal end includes an integrated circuit chip in electrical communication with said ultrasound phased array and in thermal transfer engagement with said coolant.

7. The ultrasound imaging device of claim 1 wherein said coolant is castor oil.

8. The ultrasound imaging device of claim 1 further comprising a heat sink in contact with said coolant to define a thermal management sub-system configured to transfer heat that is generated within said distal end.

9. The ultrasound imaging device of claim 8 wherein said heat sink is a passive component in contact with a source of said heat generated within said distal end, said fluid flow path being configured to flow said coolant to carry said heat away from said heat sink, said coolant being physically isolated from said source of said heat.

10. The ultrasound imaging device of claim 8 wherein said heat sink is a passive component that is at said proximal end of said fluid flow path, such that said heat sink dissipates said heat from said coolant.

11. The ultrasound imaging device of claim 8 wherein said thermal management sub-system further includes a source of air flow along said heat sink, thereby providing convection cooling of said heat sink.

12. The ultrasound imaging device of claim 8 wherein said thermal management sub-system further includes a TEC connected to said heat sink to carry heat away from said heat sink via said TEC.

13. The ultrasound imaging device of claim 1 further comprising an electrical cooling mechanism enabled to reduce the temperature of said coolant.

14. The ultrasound imaging device of claim 1 further comprising a fluid pump connected to said fluid flow path.

15. The ultrasound imaging device of claim 14 wherein said pump is controlled to induce flow of said coolant in discontinuous spurts.

16. The ultrasound imaging device of claim 14 further comprising at least one valve to control input and output of said coolant.

17. The ultrasound imaging device of claim 16 further comprising a bleeder valve controlled to selectively release a quantity of said coolant based upon detection that a volume of said coolant within said distal end exceeds a predetermined volume.

18. The ultrasound imaging device of claim 1 wherein said distal end includes an epoxy barrier along a face which is intended to contact a body into which ultrasound energy is to be directed.

19. The ultrasound imaging device of claim 1 wherein said fluid flow path is directed to transfer heat to blood of a body into which ultrasound energy is to be directed.

20. The ultrasound imaging device of claim 1 wherein said fluid flow path includes a single lumen between said proximal end and said distal end, said fluid flow path further including flow control to alternate the direction of flow.

21. The ultrasound imaging device of claim 1 further comprising an active thermal control system that includes a temperature monitor and electrical components which are responsive to said temperature monitor and electrical rate and heat transfer on a basis of temperature.

22. An ultrasound system comprising:
an ultrasound probe having an ultrasound phased array which generates electrical signals that are responsive to ultrasound energy;
a base unit having processing enabled to form display information from said electrical signals; and
an active thermal management sub-system that includes a lumen and a supply of cooling fluid, said lumen extending between said ultrasound probe and said base unit to carry said cooling fluid therebetween, said active thermal management sub-system being automated to provide temperature regulation.

23. The ultrasound system of claim 22 wherein said active thermal management sub-system further includes a Thermo Electric Cooler (TEC).

24. The ultrasound system of claim 23 wherein said TEC is connected to automated control to be adjusted on a basis of detecting temperature change, thereby achieving said temperature regulation, said active thermal management sub-system including a temperature monitor device for detecting said temperature change.

25. The ultrasound system of claim 23 wherein said active thermal management sub-system includes a fluid flow path of said cooling fluid and includes a thermal flow path that includes said TEC and at least a portion of said fluid flow path.

26. The ultrasound system of claim 22 further comprising a mechanical shaft connecting said base unit to said ultrasound probe, such that steering of said ultrasound probe is enabled, said lumen extending through said mechanical shaft.

27. The ultrasound system of claim 22 wherein said active thermal management sub-system includes a pump and valves that are controlled to determine flow rates and flow directions of said cooling fluid.

28. The ultrasound system of claim 22 wherein said active thermal management sub-system includes a heat sink in thermal transfer engagement with said cooling fluid.

29. The ultrasound system of claim 22 wherein said active thermal management sub-system includes a fluid bath within said ultrasound probe, said ultrasound phased array being in contact with said fluid bath.

30. The ultrasound system of claim 29 wherein said fluid bath is in thermal transfer engagement with said cooling fluid via a TEC.
